(19) United States
(12) Patent Application Publication
(10) Pub. No.: US 2004/0236231 A1
(43) Pub. Date: Nov. 25, 2004

(54) Light catheter for illuminating tissue structures

(75) Inventors: David R. Knighton, Minneapolis, MN (US); Vance D. Fiegel, New Brighton, MN (US)

Correspondence Address: POPOVICH, WILES & O'CONNELL, PA, 650 THIRD AVENUE SOUTH, SUITE 600, MINNEAPOLIS, MN, 55402, US

(73) Assignee: Embro Corporation

(21) Appl. No.: 10/445,389

(22) Filed: May 23, 2003

(51) Int. Cl. A61B 5/00; A61B 18/18

(52) U.S. Cl. 600/476; 606/15

(57) ABSTRACT

A system for the illumination of tissue structures including tubular structures within the body and a method for the use of the system. The system includes a transparent, biocompatible catheter and a light source.

### FIELD OF THE INVENTION

This invention relates to a catheter used to illuminate tissue structures within the body of a patient. In particular, this invention relates to a catheter illuminated along its entire length and methods of using the catheter to illuminate tissue structures within the patient's body.

### BACKGROUND OF THE INVENTION

During surgical procedures, it is desirable to see structures in a body. For example, it is desirable for surgeons to view various tubular tissue structures, such as veins, arteries, bile ducts, ureters, fallopian tubes, vas deferens, arterial tubes, the colon or small intestine in order to determine the condition of the tubular tissue structure, to repair or treat the tubular tissue structure, or to remove the tubular tissue structure because it is diseased or to be used for transplantation elsewhere in the patient's body or in another patient's body.

Vein harvesting is an example of one procedure which requires a surgeon to view a tubular tissue structure. Vein harvesting is commonly done in connection with coronary artery bypass surgery. The greater saphenous vein is a subcutaneous vein which is often used for coronary artery bypass grafting, infra-inguinal bypass grafting and vein-vein bypass grafting. Other vessels may also be used including the internal mammary artery, the radial artery, and/or the lesser saphenous vein.

Previously, in order to examine a tubular tissue structure or to harvest it, it has been necessary to make an incision along the full length of the vein section to be removed. The vein is then freed by severing and ligating the branches of the vein, after which the section of the vein can be removed from the patient. The full-length incision must then be closed, for example by suturing or stapling. Obviously, the harvesting of the vein in this manner leaves disfiguring scars that are cosmetically undesirable. Additionally, the large incision creates a risk of infection to the patient and may not heal properly, especially with those patients who have poor circulation in their extremities. Such an incision may create a chronic non-healing wound, requiring significant and costly medical treatment.

U.S. Pat. No. 5,772,576 (Knighton et al.) describes a device and method for vein removal. The device has one or more lumens extending through a body portion. One lumen is sized to accommodate a blood vessel and at least one tool for use in removing the vessel. The device may also include viewing means so that the operator may remotely view an area adjacent the distal end of the body portion. The device protects the vessel being removed from damage by the tools used in the procedure, which is critical since the blood vessel is destined for reuse (as in arterial bypass). In addition, a single operator can use the device.

Devices for harvesting a section of a blood vessel without creating a full-length incision include those described in U.S. Pat. No. 6,558,313 (Knighton), incorporated herein in its entirety by reference. Knighton describes an expandable hood that makes a workspace for extraction of the vein and an extendible or telescoping device having desired tools at its distal end. The tools are activated at the proximal end of the telescoping device. The method comprises illuminating the dissection area via a light catheter that is inside the lumen of a blood vessel and deploying the telescoping device to the length desired to dissect the vein from surrounding tissue. The light catheter described herein is suitable for use in the vein harvesting procedure described in this patent.

A need in the art exists for viewing tissue structures within a patient's body to locate the tissue structure and to determine if repair, treatment or removal is necessary. Most desirable, a method for doing this would be minimally invasive so as to avoid damage to bodily tissues and prolonged recovery times.

### SUMMARY OF THE INVENTION

This invention is a system for the localization of tissue structures including tubular structures within the body and a method for the use of the system. The system includes a transparent, biocompatible catheter and a light source. The system can also be used to photoactivate chemicals in localized tissue structures and to light active chemotherapeutic agents in selected tissue.

In a first embodiment the invention is a tissue illumination system comprising an elongate catheter including a light transmitting portion having a distal end and a proximal end and an outer surface between the distal and proximal ends, the distal end having a light reflective member. The system further includes a light source connected to the proximal end of the light transmitting portion, the light transmitting portion and reflective member being configured to disperse light provided from the light source along the outer surface of the light transmitting portion with an intensity sufficient to illuminate the tissue. The light transmitting portion may comprise at least one glass fiber, or at least one bundle of glass fibers. The bundle of glass fibers may include at least one fiber having a first length and at least one fiber having a second length, the first length being different from the second length. The light transmitting portion may include an outer transparent coating or an outer transparent sheath.

In another embodiment the invention is a method of illuminating a tissue structure in a body comprising inserting an elongate light transmitting element having distal and proximal ends into the body adjacent or within the tissue structure and illuminating the light transmitting element between the proximal and distal ends with light having an intensity sufficient to illuminate the tissue structure.

In a further embodiment the invention is a method of photoactivating a chemical agent which has been delivered to a tissue structure within a body comprising inserting an elongate light transmitting element into the body adjacent or within the tissue structure and illuminating the tissue structure with light transmitted from the light transmitting element, the transmitted light having properties selected to photoactivate the chemical agent in the tissue structure.

In another embodiment the invention is a method of activating a chemotherapeutic agent which has been delivered to a tissue structure within a body with light comprising inserting an elongate light transmitting element into the body adjacent to or within the tissue structure and illuminating the tissue structure with light from the light transmitting element, the transmitted light having properties selected to activate the chemotherapeutic agent in the tissue structure.

In another embodiment the invention is diagnosing a condition of a tissue structure within a body comprising inserting an elongate light transmitting element into the body adjacent to or within the tissue structure, delivering a diagnostic agent to the tissue structure and illuminating the tissue structure with light from the light transmitting element, the transmitted light having properties selected to activate the diagnostic agent.

### BRIEF DESCRIPTION OF THE DRAWINGS

FIG. 1 is a partial cross-sectional view of a portion of a patient's body showing insertion of the catheter of this invention.

FIG. 2A is a side view of one embodiment of this invention.

FIGS. 2B and 2C are partial perspective views of other embodiments of the catheter and light source of this invention.

FIGS. 2D and 2E are partial perspective views of fiber bundles.

FIGS. 3A to 3C are partial side views of the catheter of this invention illustrating light refraction therein.

FIG. 4 is a perspective view of an alternate embodiment of the catheter of this invention

### DETAILED DESCRIPTION OF THE PREFERRED EMBODIMENTS

As used herein, the term “tubular tissue structure” includes veins, arteries, bile ducts, ureters, urethras, fallopian tubes, vas deferens, arterial tubes, the colon or small intestine and any other similar tissue formation that is generally tubular in structure. The term “tissue structure” includes all tissue encompassed by the term “tubular tissue structure” plus any other tissue within a patient's body such as tumors or aneurysms.

The terms “distal” and “proximal” as used herein refer to the method of use of the system. “Proximal” refers to a location closer to the physician and “distal” refers to a location farther from the physician.

To use the light catheter described herein, the physician inserts the catheter into a tubular structure (e.g., the urethra) and advances the catheter to the desired region. The light source is activated to illuminate the tissue structure which the physician desires to view, locate or photoactivate. The catheter can be used in conjunction with imaging systems known in the art to assist the physician in placing the catheter at the desired region, or the catheter may be placed visually. That is, light from the catheter typically is sufficient to illuminate the structure and obtain proper placement. Some tissue structures will be accessed percutaneously via one or more incisions that allow the catheter to be advanced into a tubular tissue structure. The light catheter may be made in different sizes depending on the application for which it is to be used. In some procedures (e.g., coronary by-pass surgery), a guidewire is in place, and the catheter is provided with a guidewire lumen allowing it to be advanced over the guidewire to the desired location. A guidewire may be used to navigate the greater saphenous vein, for example. Tumor localization also can be done by placing a guide wire into a tumor either by percutaneous insertion or by insertion through the vasculature. In other procedures, no guidewire is required to navigate the tubular structure, such as vasculature. For those procedures the catheter may be constructed without a guidewire lumen.

In either case, the light source may be activated while the catheter is being advanced to assist in guiding the catheter to a desired region. Alternatively, the catheter can be advanced to the desired location before the light source is illuminated. The light catheter may be provided with a fiber optic cable connected to a monitor so that the physician can see the area. In some cases, however, during surgery, the light from the catheter will enable the physician to see a tissue structure without the aid of a separate fiber optic viewing device. During complex surgery, especially reoperative surgery, many tissue structures are difficult to identify in hardened scar tissue. By illuminating these structures with the light from the catheter, they can be more easily identified and protected from damage during dissection. For example, during surgery on the retroperitoneum, locating the ureters is important to protect them from injury during the dissection. This invention is used by inserting the light catheter up the ureter from the bladder pre-operatively. Then, during the retroperitoneal surgery, the illuminated ureters would be visible through the retroperitoneal tissues denoting their location, so injury during dissection would be prevented.

The catheter is also useful for the illumination and identification of structures deep in tissue such as tumors or aneurysms. It is standard practice to identify a tumor or small aneurysm radiologically. Then a surgeon operates to remove or repair the structure. Using this catheter, the structure is localized by standard interventional radiological techniques using guidewires. The catheter of this invention is threaded over the guidewire and left in place. The patient is then taken to the operating room and the light catheter is illuminated, providing a visual guide to the surgeon during the procedure.

Though the primary use of the catheter of this invention is envisioned as being in conjunction with visible light, light of various wavelengths may be used and are known in the art to achieve various desired results. For example, light in the infrared has a higher penetration of bodily tissues.

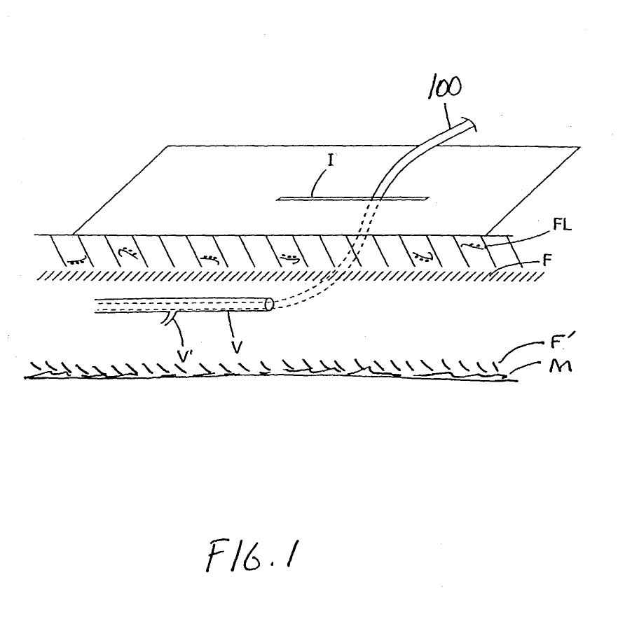

FIG. 1 illustrates the use of the catheter of this invention during a vein harvesting procedure. The light catheter is inserted into the greater saphenous vein V either through an incision or percutaneously. This vein typically has side branches V′. If an incision is going to be used, after preparation of the incision site, the physician makes small incision (I) (about 3 cm long) over the blood vessel or vein through the skin (S) and through various layers such as scarpa's fascia (F) and subcutaneous fat layer (FL). Underneath the greater saphenous vein is fascia (F′) and muscle (M). If the percutaneous method is going to be used, the physician places a needle through this and into the lumen of the greater saphenous followed by a guidewire. The light catheter moves proximally along the guidewire, thus illuminating a length of the vein. This method can be used to visualize the course and branches of the greater saphenous vein or during harvesting of the saphenous vein, as described in U.S. Pat. No. 6,558,313 (Knighton).

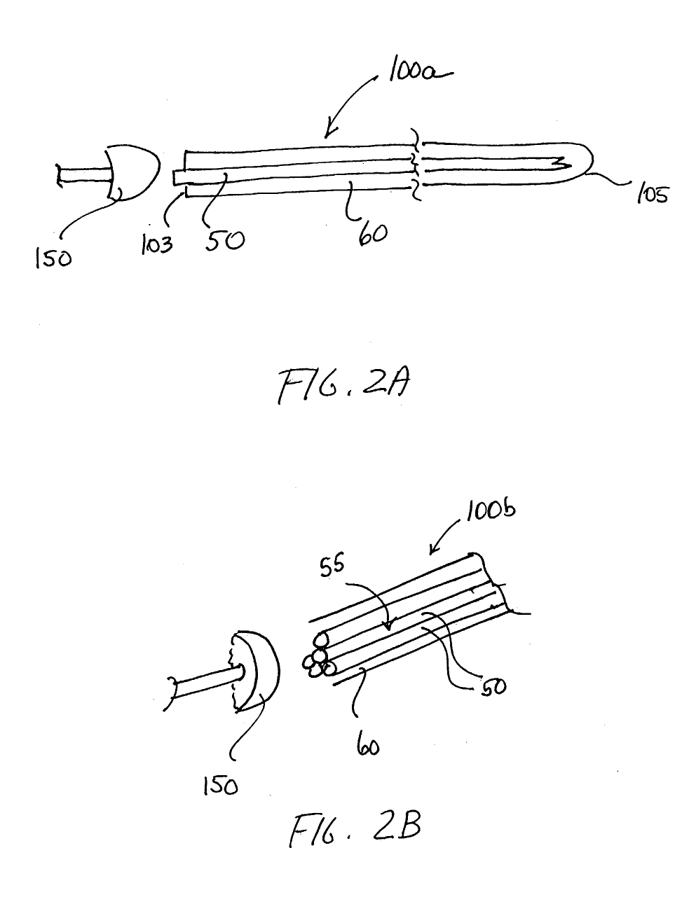

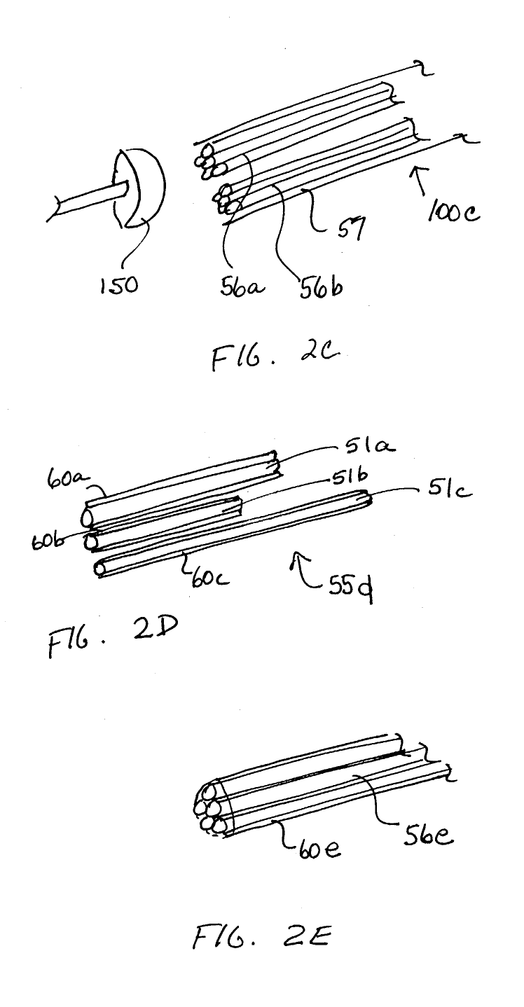

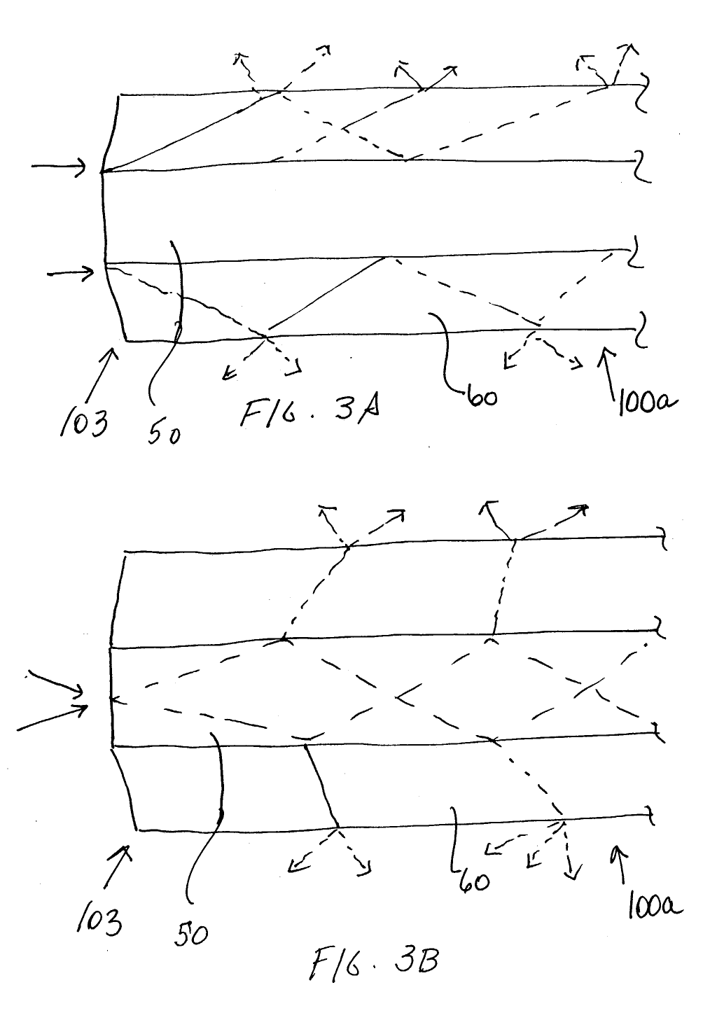

FIGS. 2A to 2E illustrate different embodiments of the catheter of the present invention. In all embodiments, the catheter comprises one or more optical fibers. For example, catheter 100a, shown in FIG. 2A, comprises central glass fiber 50 having a transparent coating or layer 60 on it. This coating or cladding is selected so as to be transparent to light of the desired wavelength. The coating or cladding is also flexible and unbreakable. In a preferred embodiment, the coating comprises biocompatible material. A suitable material for coating 60 comprises polyimide, which has been shown to be a biocompatible material offering high integrity against cracking or breaking. FIG. 2B shows another embodiment of the light catheter similar to the catheter of FIG. 2A except that catheter 100b comprises a bundle of glass fibers 55. When the catheter comprises one or more bundles of fibers (FIGS. 2B and 2C), the fibers in the bundle can be of varying lengths. This serves to distribute the illumination from the catheter along the entire length of the catheter.

FIG. 2C illustrates catheter 100c comprising multiple bundles of fibers within sleeve 57. For the sake of clarity, only two bundles 56a and 56b are illustrated, though any number of bundles can be used. FIG. 2D illustrates bundle 55d having fibers 51a, 51b, and 51c of different lengths. Any number of fibers can be used to form a bundle. In addition, FIG. 2D illustrates that each fiber may have its own coating (60a, 60b, and 60c, respectively). This is in contrast to FIG. 2E, in which the fibers are not individually coated but the fiber bundle 56e is coated with layer 60e.

The fibers in catheters 100b and 100c may each be coated with a transparent unbreakable coating, the fiber bundle may be coated with this coating, or the bundle of fibers may be placed in a sleeve, such as sleeve 57 in FIG. 2C.

In a preferred embodiment, fiber 50 is approximately 60 micrometers (microns) in diameter. Coating 60 is approximately 40 microns thick, and is optimized to enhance scattering of the light passing through glass fiber 50.

Proximal end 103 of catheter 100a is operably connected to light source 150. Distal end 105 of catheter 100a is constructed so that light is reflected back through central glass fiber 50. Preferably both the glass fiber and the coating comprise low atomic weight materials to minimize interference with X-rays.

The fibers in catheters 100b and 100c may each be coated with a transparent unbreakable coating, the fiber bundle may be coated with this coating, or the bundle of fibers may be placed in a sleeve, such as sleeve 57 in FIG. 2C.

Light source 150 includes any suitable external light source that creates a disperse, non-collimated pattern of light. Such include fluorescent or incandescent lights, light emitting diodes, laser diodes, lasers, chemiluminescent light sources, and other equivalent light sources as will be familiar to those of skill in the art.

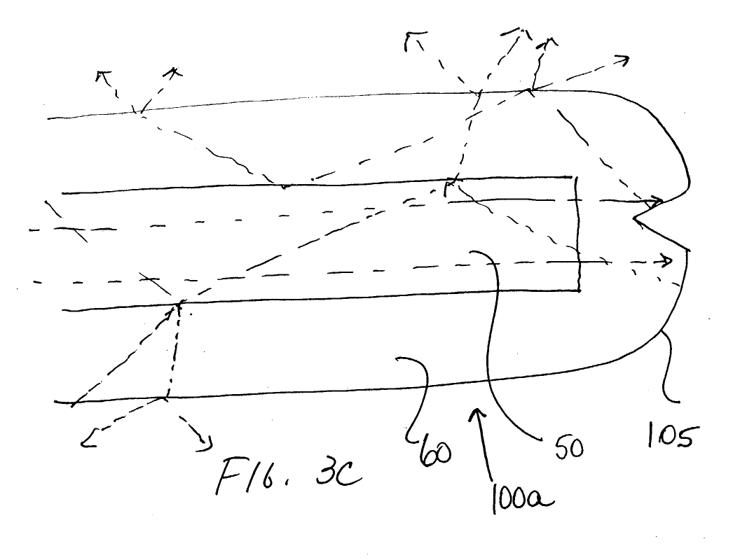

Fiber 50 and coating 60 are transparent to the light emanating from the light source. As illustrated in FIGS. 3A to 3C, light having a high incident angle disperses from the fiber, illuminating the coating uniformly. Light with low incident angles travels through the fiber to the end and is reflected back at higher angles through the coating layer.

There are factors that can be manipulated or optimized in order to ensure uniform lighting along the length of the catheter. For example, the wavelength of the light from the light source can be selected to optimize uniform lighting. The parameters or specifications of the optical system can be selected to create the light patterns (FIGS. 3A to 3C) in specific intensity ratios. The specific structure and coating at the reflective distal end of the catheter can be selected to achieve specific ratios for light patterns as disclosed in FIGS. 3A and 3B. Imperfections in the outer coating material and surface can be created to optimize light dispersion.

FIGS. 3A to 3C illustrate various ways in which light travels through catheter 100a. In FIG. 3A, light is flooded at a variety of angles into the coating 60 of the catheter at proximal end 103. Light refracts through the coating and out of the catheter at various points along the length of the catheter.

In FIG. 3B, the light enters fiber 50 at the proximal end of the catheter at an angle greater than the numerical aperture of the fiber, and refracts through the fiber and into the coating. In FIG. 3C, distal end 105 of the catheter is illustrated. In FIG. 3C, light enters the fiber at an angle less than the numerical aperture of the fiber and is guided through the fiber to the reflective distal end of the fiber. The distal end has an optical reflection system that includes retroreflective materials known in the art, such as microspheres, corner cubes, or dispersive films. These materials cause the light to reflect back into the catheter and through the fiber and its coating. Uniform lighting along the length of the catheter can be obtained through several material and geometric designs. For example, the degree of scatter of the light can be controlled by varying the index of refraction of the fiber and its coating. In addition, various length fibers can be combined in a bundle to create new light source initiation spots for the light scattering.

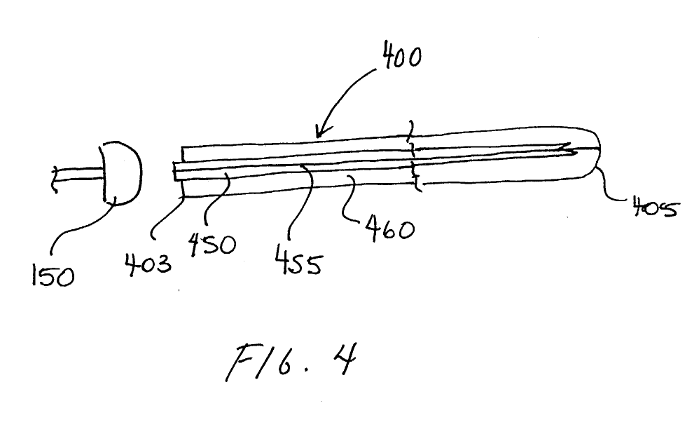

FIG. 4 illustrates an alternate embodiment of the catheter of this invention. This catheter is similar to the catheter shown in FIG. 2, except that it is provided with a guidewire lumen. In some procedures where the catheter is to be placed in specific arteries, ureters, bile ducts, or for localization of tumors, aneurysms or abscises it is advantageous to navigate the catheter over a guidewire to the desired location. Catheter 400 comprises glass fiber 450 coated with layer 460. Proximal end 403 of catheter 400 is operably connected to light source 150, and distal end 405 reflects light back into the catheter. Catheter 400 includes guidewire lumen 455 which is sized to slideably receive a guidewire. Of course catheter 400 also may comprise one or more fiber bundles, (as shown in FIGS. 2B and 2C, respectively), in which a guidewire lumen is provided within a bundle or between bundles. The lumen need not be symmetrically disposed.

In addition to the methods of using the light catheter of the present invention for harvesting vessels the catheter can be used in various other procedures to locate and aid in the treatment, repair or removal of other tissue structures. For example, the light catheter can be used to localize tumors, arteriovenous malformations, abscesses or other soft tissue abnormalities which can be localized using radiologic procedures. To localize a tumor, for example, a radiologist can visualize the tumor using a number of different imaging techniques. A guidewire can then be placed either directly to the tumor or through an artery or vein that feeds or drains the particular lesion. The guidewire is left in place in the patient. In the operating room, the light catheter is inserted over the guidewire to the lesion. When connected to a light source, the light catheter would illuminate the lesion and provide a visual guide for the surgeon to follow. This would allow localization and identification of the lesion even if it resided deep within a solid organ such as a kidney or liver.

Another use is localization of small arterial aneurysms for surgical repair. The steps in this procedure include placing a guidewire through an artery or vein to the aneurismal blood vessel. When taken to surgery, the light catheter is placed over the guidewire and the aneurysm is illuminated by the light catheter, allowing the operating surgeon to localize the aneurysm, thus aiding in repair.

The illumination catheter can be used to illuminate, or irradiate with light, chemical substances which have been introduced into tissues for diagnostic or therapeutic purposes. Therapeutic applications include photodynamic therapy using photosensitizing agents and photoactivation of drugs, biologics, receptors, and affinity reagents. For diagnostic application, the catheter can deliver energy, in the form of light of specific wavelengths, for the excitation of reporter molecules such as fluorescent compounds.

One such additional use for the system and catheter of this invention is in the photoactivation of various chemicals and/or the initiation of various chemical reactions. This includes, for example, affinity agents.

Another use is in light activated chemotherapy. New chemotherapeutic agents are being developed which are activated by light. The light catheter is used to illuminate any tumor or other tissue which is to be treated with the light activated chemotherapeutic agents. The radiologist places a guidewire within the tumor or other lesion. The chemotherapeutic agents are administered and the light catheter is threaded over the guidewire to the lesion. The appropriate wavelength of light is delivered, thus activating the chemotherapeutic agents in the area of the legion. This method decreases the exposure of normal, healthy tissue to the chemotherapeutic agents.

Although particular embodiments have been disclosed herein in detail, this has been done for purposes of illustration only, and is not intended to be limiting with respect to the scope of the claims. In particular, it is contemplated that various substitutions, alterations, and modifications may be made to the invention without departing from the spirit and scope of the invention as defined by the claims.

For example, the light catheter disclosed herein may be constructed so that it does not illuminate along the entire length from proximal to distal end but only along such length of the catheter as is necessary to sufficiently illuminate the tissue structure in question for the application selected. Further, a number of the various uses for the catheter and system disclosed herein can be performed during a single procedure, either concurrently or sequentially. For example, the system can be used to localize, view and activate chemical agents within a tissue structure at the same time or sequentially. Further, the system can be used to view a tissue structure with white light provided from the light source and then to activate a chemical agent (such as a chemotherapeutic agent, affinity agent or diagnostic agent) within the tissue structure.

What is claimed is:

1. A tissue illumination system comprising:
an elongate catheter including a light transmitting portion having a distal end and a proximal end and an outer surface between the distal and proximal ends, the distal end having a light reflective member; and
a light source connected to the proximal end of the light transmitting portion, the light transmitting portion and reflective member being configured to disperse light provided from the light source along the outer surface of the light transmitting portion with an intensity sufficient to illuminate the tissue.

2. The system of claim 1 wherein the light transmitting portion comprises at least one glass fiber.

3. The system of claim 1 wherein the light transmitting portion comprises at least one bundle of glass fibers.

4. The system of claim 3 wherein the bundle of glass fibers includes at least one fiber having a first length and at least one fiber having a second length, the first length being different from the second length.

5. The system of claim 1 wherein the light transmitting portion includes an outer transparent coating.

6. The system of claim 1 wherein the light transmitting portion includes an outer transparent sheath.

7. A method of illuminating a tissue structure in a body comprising:
inserting an elongate light transmitting element having distal and proximal ends into the body adjacent or within the tissue structure; and
illuminating the light transmitting element between the proximal and distal ends with light having an intensity sufficient to illuminate the tissue structure.

8. A method of photoactivating a chemical agent which has been delivered to a tissue structure within a body comprising:
inserting an elongate light transmitting element into the body adjacent or within the tissue structure; and
illuminating the tissue structure with light transmitted from the light transmitting element, the transmitted light having properties selected to photoactivate the chemical agent in the tissue structure.

9. A method of activating a chemotherapeutic agent which has been delivered to a tissue structure within a body with light comprising:
inserting an elongate light transmitting element into the body adjacent to or within the tissue structure; and
illuminating the tissue structure with light transmitted from the light transmitting element, the transmitted light having properties selected to activate the chemotherapeutic agent in the tissue structure.

10. A method of diagnosing a condition of a tissue structure within a body comprising:
inserting an elongate light transmitting element into the body adjacent or within the tissue structure;
delivering a diagnostic agent to the tissue structure within the body; and
illuminating the tissue structure with light from the light transmitting element, the transmitted light having properties selected to activate the diagnostic agent.
